(19) United States
(12) Patent Application Publication
(10) Pub. No.: US 2024/0426757 A1
(43) Pub. Date: Dec. 26, 2024

(54) Spectroscopic Methods and Systems for the Qualitative and Quantitative Analysis of Samples

(71) Applicant: Mariposa Technology, Inc., Metarie, LA (US)

(72) Inventors: Mickal ADLER, Metarie, LA (US); John K. ROBERTS, III, Gretna, LA (US); James F. GUENTHER, Metarie, LA (US); Brian C. SMITH, Dover, DE (US)

(73) Assignee: Mariposa Technology, Inc., Metarie, LA (US)

(21) Appl. No.: 18/753,352

(22) Filed: Jun. 25, 2024

Related U.S. Application Data
(60) Provisional application No. 63/510,188, filed on Jun. 26, 2023.

(51) Int. Cl. G01N 21/65; G01N 21/01; G01N 33/94

(52) U.S. Cl. CPC G01N 21/65; G01N 21/01; G01N 33/948; G01N 2021/0137

(57) ABSTRACT

The present invention discloses improved analytical methods that use spectral quality, classification, and quantitative algorithms to properly vet date and thereby enable the use of conventional spectroscopic methods to easily, quickly, and automatically identify and quantify chemical species in samples.

### PRIORITY

This application claims the benefit of and priority to U.S. Prov. Appl. No. 63/510,188, filed Jun. 26, 2023, the entire contents of which are incorporated by reference herein.

### FIELD OF THE PRESENT INVENTION

The present invention relates generally to methods for analyzing samples using spectroscopy. More specifically, the present invention relates to the use of spectroscopic methods and a series of algorithms to identify chemical species and measure their concentrations in samples, particularly the use of Raman spectroscopy to identify and quantify chemical species in cannabis plants.

### BACKGROUND OF THE PRESENT INVENTION

Two of the most common applications for analytical chemistry methods (a) identifying the chemical species in a sample (i.e., qualitative analysis) and (b) measuring the concentrations of such species (i.e., quantitative analysis) [1, 2]. The types of analyses being performed on a sample will depend on the type of information desired. For example, qualitative analysis can answer the question, “Does this tablet contain aspirin?” whereas, quantitative analysis would answer the question, “How much aspirin is in this tablet?”

Traditionally, the techniques used to identify chemical species have differed from those used to quantify chemical species. For example, techniques such as infrared spectroscopy and mass spectrometry are generally used to identify chemical species present [2, 4], whereas chromatography is more often preferred for quantitative analysis. [5] This dichotomy in analytical technique presents a problem when one is presented with an unknown where one wants to both identify and quantify the chemical species present. Currently, such a two-pronged analysis requires the use of multiple instruments that are slow, expensive, and difficult to use. For example, solving the problem posed above with the aspirin tablet would require the steps of first grinding up the tablet of interest and extracting its contents into a solvent, then injecting the sample into a chromatograph to purify it, and finally identifying the purified components using a mass spectrometer or infrared spectrometer. As such a process inherently requires significant sample preparation, the use of multiple instruments that are expensive and difficult to use and skilled but expensive operators, it is quite expensive and time consuming.

Accordingly, given the existing drawbacks of the commercially available options noted above, there remains a consistent need in the art for a technique that can both qualitate and quantify samples in a fast, inexpensive, easy, and automated manner.

### SUMMARY OF THE PRESENT INVENTION

Spectroscopy is the study of the interaction of electromagnetic radiation with matter [9]. Spectroscopic techniques are optimal in the context of the present invention because they have long been used to identify chemical species [1] and measure concentrations in samples [2]. For example, near infrared spectroscopy has been known to be used to determine fat, protein, and moisture in a number of plant matrices [3]. Likewise, Raman spectroscopy has been used to analyze cannabis plants [7, 15].

The advantage that spectroscopic techniques have over chromatographic techniques is that spectroscopic techniques normally require little or no sample preparation, whereas chromatography requires significant weighing, extracting, dissolving, and filtering of a sample prior to analysis and further involves interfacing a spectrometer to the chromatograph to identify the chemical species present, all of which factors into a slow and expensive process that requires skilled technicians to perform the work.

A spectrum of a sample can contain all the information needed to both qualitate and quantitate a sample. For example, the peak positions in a Raman spectrum of a sample can identify the chemical species present, and the size of the peaks can disclose concentration. Traditionally a trained human being was needed to interpret the spectrum to determine the chemical species present and measure the peak heights and/or areas needed for quantitation. However, modern automation processes, and particularly computer run algorithms and artificial intelligence programs, allow for a decrease analysis time and costs

Algorithms can serve to automate many chemical analyses. For example, automated library searching can facilitate the identification of chemical species, and automated quantitative algorithms using calibration models generated using algorithms such as Partial Least Squares (PLS) can be used to determine the concentration(s) of analyte(s) in samples. However, many quantitative algorithms are matrix specific, that is they only work properly on samples of known chemical composition. For example, due to differences in sample matrices, a method developed to measure the amount of aspirin in a tablet cannot likewise serve to determine the amount of aspirin excreted in a sample of human urine. Rather, any attempt to use the former method on the latter sample would give rise to calibration inapplicability [2].

Thus, human intervention is typically required to ensure that the sample type being measured is compatible with the quantitative analysis calibration model in hand. The present invention eliminates this requirement by using a series of algorithms. For example, in the context of an illustrative embodiment of the present invention, the first step after measuring the spectrum of a sample is to apply a plurality of spectral quality algorithms to determine if the spectrum is of acceptable quality. For example, the data may be too noisy or the peak positions incorrectly measured. In a second step of an illustrative embodiment of the present invention, a plurality of classification algorithms are applied to the spectrum to determine whether the sample is of the correct class to be quantitated. If the sample is of the correct class, a plurality of quantitative algorithms can be used to determine the concentration of analyte(s) in the sample. In this way the process of determining spectral data quality, sample classification, and analyte quantitation can be automated to increase efficiency and save time and money.

Accordingly, it is therefore an objective of the present invention to provide an automated method for analyzing a sample of interest that involves the steps of:
(i) measuring a spectrum of the sample,
(ii) applying a spectral quality algorithm to determine if the spectrum is of acceptable quality,
(iii) if the spectrum is of acceptable quality, applying a classification algorithm to the spectrum to determine if the sample is of the correct class, and
(iv) if the spectrum is of the correct class, applying a quantitative algorithm to the spectrum to determine the concentration(s) of chemical specie(s) present.

U.S. Pat. No. 11,293,858 to Smith describes a method of using spectra and a plurality of quantitative algorithms to analyze cannabis samples for their THC level. However, in contrast to the method of the present invention such as exemplified above, the Smith method does not mention the use of spectral quality algorithm(s) (step iii), nor does the use of classification algorithm(s) (step iv). Critically, in the absence of a spectral quality algorithm, bad data can be analyzed, and, likewise, in the absence of a classification algorithm, incorrect quantitative results can be reported. The inclusion of such safeguard screens in the methods of the present invention constitutes a novel improvement over the disclosure of Smith.

While the present invention is not limited to a particular spectroscopy method, in preferred embodiments, the spectroscopic method is selected from among radio wave, microwave, far infrared, mid-infrared, near infrared, visible, ultraviolet, x-ray, absorption, reflection, transmission, scattering, emission, and Raman. In a particularly preferred embodiment, the spectroscopy used is a Raman spectrum.

In preferred embodiments, the spectral analyzer used is chosen from among dispersive, Fourier transform, non-dispersive, filter based, and Fabry-Perot.

In preferred embodiments, the spectral quality algorithm is chosen from the among noise level, peak-to-peak noise level, root mean square noise level, signal-to-noise ratio, and a peak position or positions compared to the peak positions of a reference standard.

In preferred embodiments, the classification algorithm is chosen from among of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor.

Classification, Kernel Approximation Classification, Ensemble Classification, Neural Net Classification, library searching, spectral subtraction, classical least squares, K-Matrix, inverted least squares, and P-matrix.

The present invention is not limited to a particular output device or connection. As such, the output device may be wired or wirelessly connected and, in preferred embodiments, can be selected from among a cellular phone, smart phone, and computer.

In particularly preferred embodiment of the present invention, the analytical method of the present invention may be directed to the assessment of a plant sample, such as a root, stem, branch, leaf, flower, or seed, more particularly a cannabis sample. Accordingly, it is a further objective of the present invention to provide an automated method for analyzing a cannabis sample by:
(i) measuring a Raman spectrum of the cannabis sample,
(ii) applying a spectral quality algorithm to determine if the Raman spectrum is of acceptable quality,
(iii) if the Raman spectrum is of acceptable quality, applying a classification algorithm to the Raman spectrum to determine if the sample is of the correct class, and
(iv) if the spectrum is of the correct class, applying a quantitative algorithm to the spectrum to determine the concentration(s) of chemical specie(s) present, particularly the concentration of any Δ-9 tetrahydrocannabinol present.

These and other aspects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims.

### BRIEF DESCRIPTION OF THE FIGURES

FIG. 1 The Raman spectrum of a cannabis leaf.

FIG. 2A flow chart of the present invention

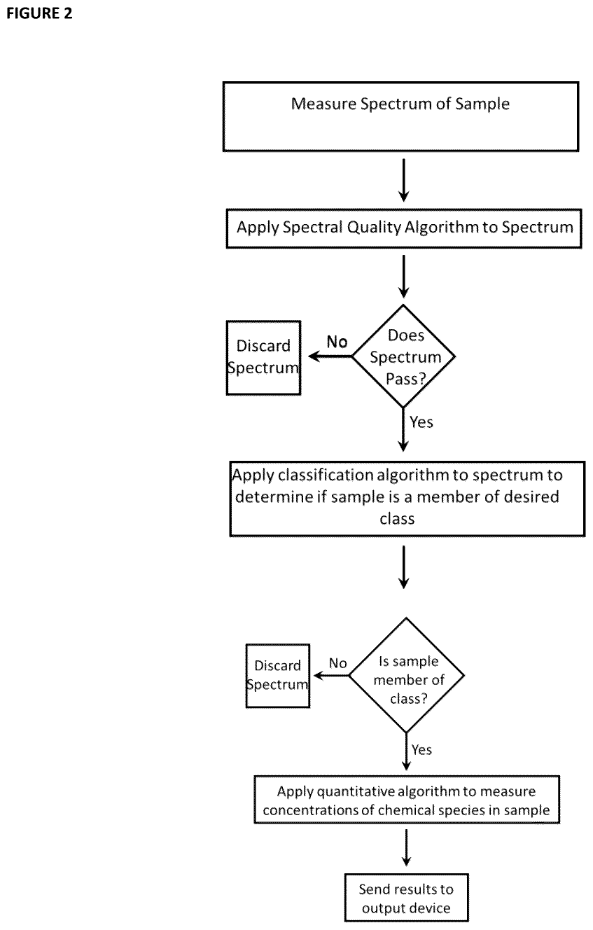

### DETAILED DESCRIPTION OF THE PREFERRED EMBODIMENTS

Unless otherwise defined herein, scientific and technical terms used in the present disclosure shall have the meanings that are commonly understood by those of ordinary skill in the art. In case of conflict, the present specification, including definitions, will control.

The term “a” or “an” entity refers to one or more of that entity; for example, “a vector,” is understood to represent one or more vectors.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C;

A, B, or C; A or C; A or B; B or C; A and C; A and B; B and C; A (alone); B (alone); and C (alone).

Unless otherwise required by context, singular terms shall include pluralities and plural terms shall include the singular.

In the context of the present invention, the phrase “chemical species” encompasses but is not limited to atoms, molecules, ions, elements, and elementary particles.

The process of identifying chemical species in a sample referred to in the art as “qualitative analysis”. Likewise, the process of performing a qualitative analysis is known in the art “qualitation”. In the context of the present invention, the terms “identify” and “qualitate” are considered to be synonymous and thus used interchangeably.

In contrast to “qualitative analysis”, the process of measuring concentrations is known in the art as “quantitative analysis”. In the context of the instant invention, term “concentration” refers to the amount of chemical species in a sample or any property that depends upon concentration, examples of which include but are not limited to, viscosity and octane number, i.e., sample properties related to chemical species concentration [3]. In the context of the present invention, the chemical species being identified or quantified is referred to as an “analyte”.

Different types of electromagnetic radiation are defined by wavelength, wavenumber, and frequency amongst other properties discussed in the literature [9]. In the context of the present invention, the term “light” encompasses any and all types of electromagnetic radiation.

### Spectroscopy:

Spectroscopy uses electromagnetic radiation to analyze samples. For the purposes of the present invention, any type of electromagnetic radiation may be used including, but not limited to, radio waves, microwaves, far infrared, mid-infrared, near infrared, visible, ultraviolet, lasers, and x-rays.

In the particle model of light [2], beams of electromagnetic radiation can be thought of as containing massless particles called photons. When a beam of light interacts with a sample, many different types of phenomena can occur, examples of which include, but are not limited to, absorption, transmission, emission, reflection, refraction, diffraction, and scattering. The photons that have interacted with a sample can be collected and their properties determined. This is typically done with a spectral analyzer or “spectrometer”. For the purposes of the present invention the types of spectral analyzer that can be used in the context of the present invention include, but are not limited to, dispersive, Fourier transform, non-dispersive, filter based, and Fabry-Perot.

For the purposes of the present invention the spectral properties that can be measured by a spectral analyzer include but are not limited to. wavelength, wavenumber, frequency, intensity, absorbance, reflectance, reflectivity, transmittance, percent transmittance, emission, emissivity, scattering intensity, counts, Raman scattering intensity, and arbitrary intensity. A spectrum can be a two-dimensional plot with, for example, a measure of light intensity on the y-axis and some property of light on the x-axis. For example, a Raman spectrum can be a plot of scattering intensity on the y-axis versus wavenumber on the x-axis.

Spectroscopy requires a source of electromagnetic radiation. For the purposes of the present invention, the types of light sources that may be used to measure spectra include, but are not limited to, broad band sources, narrow band sources, modulated light sources, non-modulated light sources, and lasers. Critically, the context of the present invention any type of spectroscopy may be used.

To measure a spectrum, electromagnetic radiation is caused to impinge on a sample. Photons that have interacted with the sample are collected, and are then analyzed by a spectral analyzer to measure a spectrum of the sample.

### Raman Spectroscopy:

Raman scattering occurs when photons are inelastically scattered by molecules [6, 7]. Because of the law of conservation of energy, the amount of energy lost by the inelastically scattered photons must equal the amount of energy gained by the molecule from which it is scattered. Typically, the collision between a photon and a molecule excites vibrational modes of the molecule. For example, if a methyl (CH₃) group has an asymmetric C—H stretching vibrational energy level at 2962 cm⁻¹ [1], a photon of higher energy may collide with the molecule containing the methyl group, lose 2962 cm⁻¹ of energy and excite the asymmetric stretch of the methyl group. In Raman spectroscopy, the inelastically scattered photons are gathered, analyzed by a spectral analyzer, and then plotted with appropriate units. When plotted with Raman Shift on the x-axis, the peak positions in a Raman spectrum represent the energy of vibrational energy levels excited in a molecule. This gives chemical information, including the identity of functional groups, which can be used to identify molecules and measure their concentrations in samples.

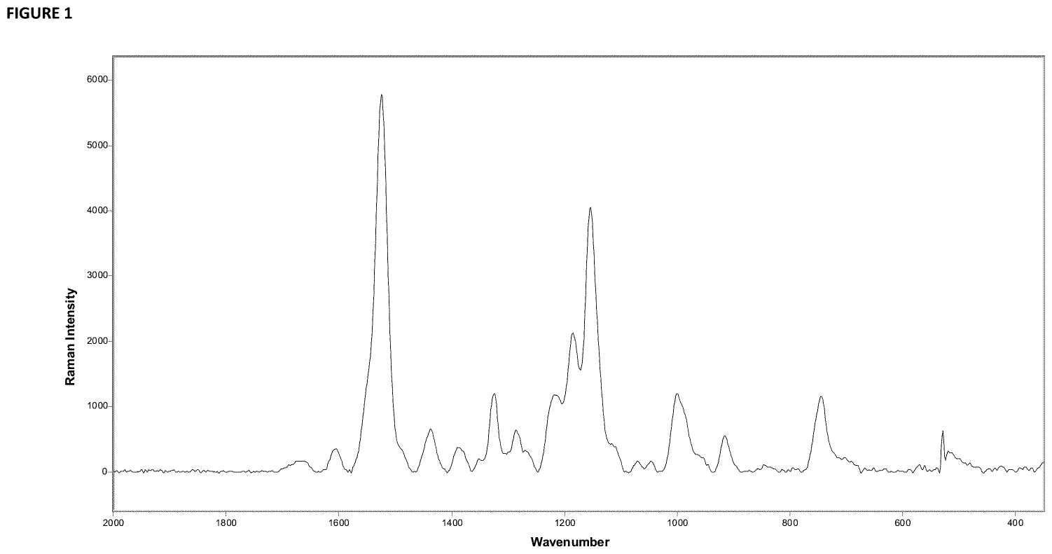

An advantage of Raman spectroscopy is that there is very little sample preparation. For many samples, it is simply a matter of illuminating a sample with a beam of electromagnetic radiation, analyzing the energy of the scattered photons, and then plotting the Raman spectrum. An example of the Raman spectrum of a marijuana leaf is seen in FIG. 1.

### Spectral Quality Algorithms:

Like the measurement of any data, some data are good and others are bad, and this is true of spectra as well. Accordingly, it is important to weed out bad spectra in any analysis to save time and avoid the generation of inaccurate results. Spectra are 2-dimensional data objects composed of data on an x-axis and a y-axis. The x-axis is frequently plotted in units that are some property of light, such as wavelength, wavenumber, or frequency. The y-axis is typically plotted in units of measured light intensity, such as intensity, absorbance, reflectance, reflectivity, transmittance, percent transmittance, emission, emissivity, scattering intensity, counts, Raman scattering intensity, and arbitrary intensity. Of course, like any measurement, both of these sets of sets of data will contain a margin of error. Thus, spectral quality algorithms exist to measure the accuracy of the x-axis data and y-axis data in spectra.

One way of ensuring the accuracy of the x-axis data in a spectrum is to compare the peak positions in a sample spectrum to the peak positions in the spectrum of a known standard reference material. For example, in infrared spectroscopy polystyrene is a standard reference material whose peak positions are well studied and known [8]. The spectrum of polystyrene then can be compared to the spectrum of a sample to see if the peak positions have been measured accurately or not [9]. Another way of vetting the x-axis data in a spectrum is to use an algorithm that specifies that specific peaks must be present in a spectrum within a specific margin of error. For example, in Fourier transform infrared spectroscopy (FTIR), the margin of error for peak positions is known to be ±½ the instrumental resolution setting used to measure a spectrum [9]. Thus, a spectrum measured at 4 cm−1 instrumental resolution will have a peak position margin of error of ±2 cm⁻¹. In an illustrative embodiment, a spectral quality algorithm may consist of a plurality of known peak positions that are then compared to the peak positions in a sample spectrum to see if they are within a pre-determined margin of error.

In another embodiment, Raman spectra can be used as the reference standard. More particularly, since the Raman spectra of cannabis plants a plurality of peaks is known to be present at specific wavenumbers, a spectral quality algorithm can be used to test whether said peaks are present at the correct positions to thereby assure that the x-axis data have been accurately measured.

The margin of error in y-axis data in a spectrum is often referred to as “noise”. There are a number of ways of measuring noise in a spectrum. For example, a parameter known as “peak-to-peak noise” can be measured by taking the highest and lowest noise points in a given spectral region, subtracting them from each other, and then taking the absolute value of this quantity [9]. Other algorithms that can be used to measure spectral noise include the root mean square method.

A measure of spectral quality is called the signal-to-noise ratio (SNR) [9]. This quantity is calculated using equation 1:

$$SNR = Signal / Noise \qquad ( 1 )$$

The “signal” is typically measured as the size of a specific peak in a spectrum, whereas the “noise” is measured in a specific spectral baseline region free of peaks using a noise measuring method such as one of the ones described above. The higher the SNR, the better the quality of a spectrum. Thus, an SNR measurement can be used as a specific measure of spectral quality. For example, whether the SNR of an individual peak is above or below a specific threshold can be used to accept or reject a spectrum. Alternatively, the SNR for a plurality of peaks can be compared to a plurality of thresholds to accept or reject a spectrum. In an illustrative embodiment, the SNRs for a plurality of peaks are measured and compared to a plurality of pre-determined SNR thresholds to determine if a spectrum is of appropriate quality or not.

Illustrative spectral quality algorithms that find utility in connection to the present invention include, but are not limited to, noise level, peak-to-peak noise level, root mean square noise level, signal-to-noise ratio, and a plurality of peak positions compared to the peak positions in a reference standard. For example, a measured noise level can be compared to a pre-set noise level threshold, and if the measured noise is above the noise level threshold the spectrum is not acceptable, whereas if the measured noise is below the noise level threshold it is acceptable. Similarly, the SNR can be used to determine if a spectrum is acceptable. For example, the SNR of a spectrum can be measured using a plurality of peaks and compared to a plurality of pre-set SNR thresholds. If measured SNR(s) are below a one or more SNR thresholds the spectrum may not acceptable, whereas if it is above the SNR threshold it is acceptable. As stated above, SNRs are calculated from individual peaks in a spectrum. In the context of the present invention one or more peaks with one or more SNR thresholds may be used to determine spectrum acceptability.

To ascertain whether the x-axis of a spectrum has been measured accurately, it may be necessary to compare the peak positions in a measured spectrum to those of a known reference. A peak position accuracy threshold may be used for this purpose. For example, if a peak position accuracy threshold is ±5 cm⁻¹, if the peak position of the peak of interest is within this threshold the spectrum is acceptable, and if it is outside this threshold then it is not acceptable. One or more peaks may be used for this purpose. However, all these examples are meant for illustrative purposes only, and thus it will be obvious to one of ordinary skill in the art that many other types of spectral quality algorithms are possible within the scope of the present invention.

### Classification Algorithms:

A spectral classification algorithm is used to determine if a sample belongs to a specific class of samples or not. For example, a spectral classification algorithm may be used to determine whether a given sample is cannabis or not. This is important because quantitative calibration models are matrix specific, that is they can only work on the types of samples from which the calibration model was built, and since it is impossible to build a quantitative model containing the spectrum of everything in the universe, quantitative models have limitations. An important job of the classification algorithm in the context of the present invention is to ensure that the matrix of the sample scanned is appropriate for analysis by a selected quantitative calibration model. To that end, a sample matrix can be characterized by the identity of the chemical species present, their concentration, pH, and physical quantities such as temperature and pressure. As an example, if a spectrum of pizza is analyzed by a calibration model built with the spectra of cannabis buds, incorrect results will be reported because the matrices of the two samples are different.

The types of classification methods that can be incorporated into the present invention include, but are not limited to, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

As an illustrative example, a measured Raman spectrum of a sample can be compared to a spectral library of pre-measured Raman spectra. If the hit quality index (HQI) is above a pre-set HQI threshold the spectrum is acceptable, whereas if it is below this threshold, it is not acceptable. This example is meant for illustrative purposes only, and it should be obvious to one of ordinary skill in the art that many other types of classification algorithms are possible within the scope of the present invention.

### Quantitative Calibration Models:

Quantitative calibration models can be applied to spectra to determine concentrations of chemical species and quantities related to chemical species concentration, such as viscosity and octane number [3]. Traditionally, a series of standard samples of known analyte concentrations are measured, a peak whose height or area that varies with analyte concentration is identified, and a calibration using the known concentration and measured peak height or area is built. The problem with this univariant approach is that a spectrally isolated peak free of interferences is necessary for this method to work. That is, this method only works if the analyte is the only chemical species that contributes to the spectral feature being analyzed. In the past, this has limited the use of quantitative spectroscopy to simple chemical systems with spectral interference free components, and made characterization of complex matrices such as cannabis buds impossible.

More recently, multivariant and statistically based chemometric algorithms have been introduced that have allowed successful spectroscopic quantitation of complex matrices, including cannabis buds [2, 10]. These algorithms use not a single spectral feature but entire spectra or spectral regions in their analyses. This allows the quantitation of specific analytes in complex matrices even if the concentration of all species in a sample is unknown or spectral interferences are present.

For the purposes of the present invention, any set of mathematical algorithms that can be used to analyze a spectrum may constitute a quantitative algorithm. Illustrative examples of quantitative algorithms suitable for use in the context of the present invention include, but are not limited to, principle components analysis, least squares, classical least square, K-matric, inverse least squares, P-matrix, partial least squares, principle components analysis, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, and library searching.

In an illustrative embodiment, the intensity (ies) of the peak(s) in a Raman spectrum can be measured, a calibration model obtained from spectra of standard samples and known concentration(s) can then be applied, and thus the concentration(s) of analyte(s) determined. However, as will be readily obvious to the skilled artisan, other types of quantitative algorithms may find utility within the context of the present invention.

### Algorithm Examples

A number of different spectral quality, classification, and quantitative algorithms fall within the scope of the present invention. The following is an illustrative but not exhaustive list of example algorithms that may find utility in connection with the present invention:
Linear Regression;
Generalized Linear Regression-Generalized linear regression models with various distributions and link functions, including logistic regression;
Stepwise Regression-Variable selection in generalized linear model using stepwise regression;
Regularization-Ridge regression, lasso, and elastic nets for generalized linear models;
Nonlinear Regression;
Nonlinear fixed- and mixed-effects regression models;
Support Vector Machine Regression;
Gaussian Process Regression-Gaussian process regression models (kriging);
Regression Trees-Binary decision trees for regression;
Regression Tree Ensembles-Random forests, boosted and bagged regression trees;
Generalized Additive Model-Interpretable model composed of univariate and bivariate shape functions for regression;
Incremental Learning-Fit linear model for regression to streaming data and track its performance;
Interpretability-Train interpretable regression models and interpret complex regression models;
Artificial Neural Networks (ANN): Computational models inspired by the structure and function of the human brain. ANNs consist of interconnected nodes (neurons) organized in layers and are capable of learning complex patterns and relationships;
Feedforward Neural Network (FNN): The most basic type of neural network, where information flows in one direction, from input to output, without any loops or feedback connections;
Multilayer Perceptron (MLP): A type of feedforward neural network with one or more hidden layers between the input and output layers. MLPs are widely used for various tasks, including classification and regression;
Convolutional Neural Networks (CNN): Specifically designed for processing grid-like data. CNNs utilize convolutional layers to automatically learn hierarchical representations from input data, enabling them to capture spatial dependencies effectively;
Recurrent Neural Networks (RNN): Designed to process sequential data, RNNs have feedback connections that allow information to flow in cycles. This enables them to maintain a memory of past inputs;
Long Short-Term Memory (LSTM): A specialized type of RNN that addresses the vanishing gradient problem by using memory cells and gating mechanisms. LSTMs are known for their ability to capture long-term dependencies in sequential data;
Gated Recurrent Unit (GRU): Another type of RNN that addresses the vanishing gradient problem. GRUs have a simplified architecture compared to LSTMs, with fewer gating mechanisms, making them computationally less expensive;
Self-Organizing Maps (SOM): Also known as Kohonen maps, SOMs are unsupervised learning models used for clustering and visualizing high-dimensional data. They organize data points into a low-dimensional grid, preserving the topological properties of the input space;
Radial Basis Function Networks (RBFN): These networks use radial basis functions as activation functions. RBFNs are often used for function approximation and interpolation tasks;
Hopfield Networks: A type of recurrent neural network that serves as a content-addressable memory system. Hopfield networks are capable of storing and recalling patterns, making them useful for associative memory tasks;
Generative Adversarial Networks (GAN): Consisting of two neural networks—a generator and a discriminator—GANs are used for generative modeling. The generator aims to generate realistic data samples, while the discriminator tries to distinguish between real and generated samples, creating a competition between the two networks;
Autoencoders: These networks are designed for unsupervised learning and dimensionality reduction tasks. Autoencoders consist of an encoder network that compresses the input data into a lower-dimensional representation and a decoder network that reconstructs the original input from the compressed representation;
Boltzmann Machines: A type of stochastic, generative neural network that utilizes a network of binary-valued neurons and can learn probability distributions over inputs. They are often used in areas like collaborative filtering, feature learning, and dimensionality reduction;
Linear Regression: A simple algorithm used for regression tasks, where the relationship between the input variables and the target variable is modeled as a linear equation;
Logistic Regression: Similar to linear regression, but used for classification tasks. It models the probability of an input belonging to a particular class;
Decision Trees: A tree-based algorithm that recursively partitions the data based on feature values. Decision trees are interpretable and can handle both classification and regression tasks;
Random Forest: An ensemble algorithm that combines multiple decision trees. It improves upon decision trees by reducing overfitting and increasing predictive accuracy;
Support Vector Machines (SVM): A supervised learning algorithm used for classification and regression tasks. SVMs find an optimal hyperplane that separates data points of different classes.
Naive Bayes: A probabilistic algorithm based on Bayes' theorem. Naive Bayes assumes that features are independent, making it computationally efficient;
K-Nearest Neighbors (KNN): A lazy learning algorithm that classifies data points based on their proximity to the nearest neighbors in the training set;
K-Means Clustering: An unsupervised learning algorithm used for clustering tasks. K-Means groups data points into k clusters based on similarity, aiming to minimize the within-cluster variance;
Principal Component Analysis (PCA): A dimensionality reduction technique that transforms high-dimensional data into a lower-dimensional space while preserving the most important information;
Partial Least Squares (PLS) Regression: Commonly used in spectroscopy for quantitating multiple analytes in complex matrices;
Orthogonal Projections to Latent Structures Discriminant Analysis (OPLS-DA);
Multivariate Curve Resolution (MCR);
Deep Learning: A subset of machine learning that focuses on training deep neural networks with multiple hidden layers;
Reinforcement Learning: A type of machine learning where an agent learns to interact with an environment to maximize a reward signal. Reinforcement learning is used in scenarios where there is no labeled dataset, and the agent learns through trial and error;
Genetic Algorithms: Inspired by the process of natural selection, genetic algorithms use evolutionary principles to solve optimization problems. They involve generating a population of candidate solutions and iteratively refining them;
Hidden Markov Models (HMM): A statistical model that represents systems with hidden states; and
Gaussian Mixture Models (GMM): A probabilistic model that represents the probability distribution of a set of data points as a mixture of Gaussian distributions. GMMs are used for clustering and density estimation tasks.

### Results Reporting and Output Devices:

For the present method to be useful, the results of the method need to be accessible to the user. Accordingly, the present invention contemplates the use of any type of output device capable of displaying text, numbers, and graphics. Thus, in the context of the present invention, illustrative examples of such output devices include, but are not limited to, cathode ray tube screens, liquid crystal displays, televisions, computer screens, cell phones, and smart phones.

In the context of the present invention, the output device is preferably configured to save an electronic copy of the spectroscopic results in any file format. Examples of such output devices capable of saving an electronic copy of the results include, but are not limited to, floppy disks, hard disks, USB drives, networks, network servers, and remote storage such as in the Cloud. An output device may also provide a paper copy of the results. Examples of paper copy output devices contemplated by the present invention include, but are not limited to, plotters and printers. Output devices suitable for use in the inventive context may incorporate one, some, or all of the above capabilities.

In the context of the present invention, the spectral analyzer and output device may be part of one unit, or they may be separate units. In either case, the results must be communicated to the output device. This can be done using a wired connection, examples of which include, but are not limited to, serial, parallel, USB, and Ethernet. Alternatively, the spectral analyzer and output device may communicate wirelessly. Examples of wireless protocols that may be used in the context of the present invention include, but are not limited to, Wi-Fi and Bluetooth.

### Illustrative Embodiments

Hereinafter, the present invention is described in more detail by reference to certain examples and preferred embodiments. However, it should be obvious to anyone of ordinary skill in the art that the details mentioned in all embodiments are for illustrative purposes only, and that many other variations of the present invention are possible while still being well within the scope of the present invention. As such, the following embodiments are meant to be enabling and for illustrative purposes only and are not meant to narrow the scope of the present invention in any fashion whatsoever. As such, methods and materials similar or equivalent to those described herein can be used in the practice or testing of the present invention.

### Example 1: Cannabis Plants

The following is an exemplary embodiment of the method of the present invention applied to the qualitative and quantitative analysis cannabis plants:

Human beings have been growing and using cannabis plants for thousands of years. For the purposes of the present invention, the term “cannabis plants” broadly encompasses all parts of the plant of species Cannabis Indica, Cannabis Sativa, and Cannabis Ruderalis, for example. Amongst the useful compounds found in cannabis plants are cannabinoids, examples of which include Δ-9 tetrahydrocannabinol (THC) and cannabidiol (CBD). In an illustrative embodiment, the method of the present invention can be used to determine the concentration of THC in cannabis plants including cannabis leaves or buds.

Current federal law in the United States allows possession of cannabis plants and cannabis plant-based materials as long as they contain less than 0.3 weight percent THC [11]. This means cannabis growers need to monitor the THC level in their plants as they are growing to make sure they are legal. In an embodiment of the present invention, Raman spectra of cannabis buds and/or leaves are measured, a spectral quality algorithm is used to insure data quality, a classification algorithm is used to make sure the sample is cannabis, and a quantitative algorithm is used to determine THC concentration to see if the sample is legal or not.

### REFERENCES

[1] Brian C. Smith, Infrared Spectral Interpretation: A Systematic Approach, CRC Press, Boca Raton, 1999;
[2] Brian C. Smith, Quantitative Spectroscopy: Theory and Practice, Elsevier, Boston, 2002;
[3] D. Burns & E. Ciurczak, Handbook of Near Infrared Analysis, Marcel Dekker, New York, 1992;
[4] J. Robinson, Undergraduate Instrumental Analysis, Marcel Dekker, New York, 1995;
[5] S. Bayne & M. Carlin, Forensic Applications of High-Pressure Liquid Chromatography, CRC Press, Boca Raton, 2010;
[6] R. McCreery, Raman Spectroscopy for Chemical Analysis, Wiley, New York, 2000;
[7] N. Goff, J. Guenther, J. K. Roberts, M. Adler, M. Dalle Molle, G. Matthews, and D. Kurouski, Molecules 27 (2022) 4978;
[8] D. Gupta, L. Wang, L. Hannsenn, J. Hsia, & R. Datla, National Institutes of Standards and Technology Special Publication 260-122, NIST, Gaithersburg MD, 1995;
[9] Brian C. Smith, Fundamentals of Fourier Transform Infrared Spectroscopy 2nd. Edition, CRC Press, Boca Raton, 2011;
[10] Brian C. Smith & C. A. Fucetola, Cannabis Science and Technology 3 (6) (2020) 24-38;
[11] 115th United States Congress, Senate Bill S.2667,” Hemp Farming Act of 2018;
[12] Brian C. Smith, U.S. Pat. No. 11,293,858;
[13] S. Higgins, R. Jessup, and D. Kurouski, Planta 85 (2022) 255;
[14] P. Larkin, IR and Raman Spectroscopy Principles and Spectral Interpretation, Elsevier, Boston, 2011;
[15] L. Sanchez, D. Baltensperger, and W. Kurouski, Anal. Chem., 92 (2020) 7733.

All publications, patent applications, patents and other references mentioned herein are incorporated by reference in their entirety.

While the invention has been described in detail and with reference to specific embodiments thereof, it will be apparent to one skilled in the art that various changes and modifications can be made therein without departing from the spirit and scope of the invention, which is defined by the claims that follow.

What is claimed:

1. A method for analyzing a sample, said method comprising the steps of:
a. measuring a spectrum of said sample by first exposing said sample to a beam of light to allow said beam of light to interact with said sample and then sending said beam of light though a spectral analyzer;
b. applying a spectral quality algorithm to the sample spectrum measured in step (a) to determine if said sample spectrum is of acceptable quality;
c. applying a classification algorithm to sample spectrum deemed of acceptable quality in step (b) to determine a correct class for said sample; and
d. identifying a quantitative algorithm suitable for the sample class determined in step (b) and applying said quantitative algorithm to said sample spectrum to determine the identity and concentration of any chemical species present in said sample.

2. The method of claim 1, wherein the spectral analyzer utilized in step (a) is selected from the group consisting of dispersive, Fourier transform, non-dispersive, filter based, and Fabry-Perot and the type of spectroscopy utilized in step (a) is selected from the group consisting of radio wave, microwave, far infrared, mid-infrared, near infrared, visible, ultraviolet, x-ray, absorption, reflection, transmission, scattering, emission, and Raman.

3. The method of claim 1, wherein the spectral quality algorithm applied in step (b) is selected from the group consisting of noise level, peak-to-peak noise level, root mean square noise level, signal-to-noise ratio, and a peak position or positions compared to the peak positions of a reference standard.

4. The method of claim 1, wherein the classification algorithm applied in step (c) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

5. The method of claim 1, wherein the quantitative algorithm identified and applied in step (d) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

6. The method of claim 1, wherein the chemical species information determined in step (d) is communicated to an output device via a wired or wireless connection, further wherein said output device is selected from the group consisting of a cellular phone, smart phone, and computer.

7. A method for analyzing samples using Raman spectroscopy, said method comprising the steps of:
a. measuring a Raman spectrum of said sample by first exposing said sample to a beam of light to allow said beam of light to interact with said sample and then sending said beam of light though a spectral analyzer;
b. applying a spectral quality algorithm to the Raman spectrum measured in step (a) to determine if said Raman spectrum is of acceptable quality;
c. applying a classification algorithm to Raman spectrum deemed of acceptable quality in step (b) to determine a correct class for said sample; and
d. identifying a quantitative algorithm suitable for the sample class determined in step (b) and applying said quantitative algorithm to said Raman spectrum to determine the identity and concentration of any chemical species present in said sample.

8. The method of claim 7, wherein the spectral analyzer utilized in step (a) is selected from the group consisting of dispersive, Fourier transform, non-dispersive, filter based, and Fabry-Perot and the spectral quality algorithm applied in step (b) is selected from the group consisting of noise level, peak-to-peak noise level, root mean square noise level, signal-to-noise ratio, and a peak position or positions compared to the peak positions of a reference standard.

9. The method of claim 7, wherein the classification algorithm applied in step (c) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

10. The method of claim 7, wherein the quantitative algorithm identified and applied in step (d) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

11. The method of claim 7, wherein the chemical species information determined in step (d) is communicated to an output device via a wired or wireless connection, further wherein said output device is selected from the group consisting of a cellular phone, smart phone, and computer.

12. A method for analyzing cannabis samples using Raman spectroscopy, said method comprising the steps of:
a. measuring a Raman spectrum of a cannabis sample by first exposing said sample to a beam of light to allow said beam of light to interact with said sample and then sending said beam of light though a spectral analyzer;
b. applying a spectral quality algorithm to the Raman spectrum measured in step (a) to determine if said Raman spectrum is of acceptable quality;
c. applying a classification algorithm to Raman spectrum deemed of acceptable quality in step (b) to determine a correct class for said sample; and
d. identifying a quantitative algorithm suitable for the sample class determined in step (b) and applying said quantitative algorithm to said Raman spectrum to determine the identity and concentration of any chemical species present in said sample.

13. The method of claim 16, wherein the spectral analyzer utilized in step (a) is selected from the group consisting of dispersive, Fourier transform, non-dispersive, filter based, and Fabry-Perot.

14. The method of claim 16, wherein the spectral quality algorithm applied in step (b) is selected from the group consisting of noise level, peak-to-peak noise level, root mean square noise level, signal-to-noise ratio, and a peak position or positions compared to the peak positions in a reference standard.

15. The method of claim 12, wherein the classification algorithm applied in step (c) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

16. The method of claim 12, wherein quantitative algorithm identified and applied in step (d) comprises one or more of the algorithms selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

17. The method of claim 12, wherein the chemical species information determined in step (d) is communicated to an output device via a wired or wireless connection, further wherein said output device is selected from the group consisting of a cellular phone, smart phone, and computer.

18. The method of claim 12, wherein said method is utilized to determine the concentration of Δ-9 tetrahydrocannabinol present in said cannabis sample.

19. The method of claim 18, wherein the classification algorithm applied in step (c) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.

20. The method of claim 19, wherein the quantitative algorithm identified and applied in step (d) is selected from the group consisting of principle components analysis, least squares, partial least squares, discriminant analysis, linear discriminant analysis, neural networks, SIMCA (Soft Independent Modeling of Class Analogies), Machine Learning and Artificial Intelligence algorithms, Multivariate Curve Resolution (MCR), Decision Trees, Nearest Neighbor Classification, Kernel Approximation Classification, Ensemble Classification, Neural Net Classification, library searching, spectral subtraction, classical least squares, K-Matrix, inverted least squares, and P-matrix.
